(19) United States
(12) Patent Application Publication
(10) Pub. No.: US 2024/0180409 A1
(43) Pub. Date: Jun. 6, 2024

(54) CAPSULE ENDOSCOPE SYSTEM AND ITS MAGNETIC POSITIONING METHOD

(71) Applicant: SHENZHEN SIBERNETICS CO., LTD., Shenzhen (CN)

(72) Inventors: Can Peng, Shenzhen (CN); Liu Liu, Shenzhen (CN)

(73) Assignee: SHENZHEN SIBERNETICS CO., LTD., Shenzhen (CN)

(21) Appl. No.: 18/550,992

(22) PCT Filed: Apr. 29, 2021
(86) PCT No.: PCT/CN2021/091222

§ 371 Date: Sep. 18, 2023

(30) Foreign Application Priority Data
Mar. 18, 2021 (CN) 202110290148.3

(51) Int. Cl. A61B 1/04; A61B 1/00

(52) U.S. Cl. CPC A61B 1/041; A61B 1/00009; A61B 1/00158; A61B 2562/0219

(57) ABSTRACT

Some embodiments of the disclosure describe a capsule endoscope system. In some examples, the system includes a capsule endoscope, a magnetic control device, and a processing device. The capsule endoscope has a first magnet, an IMU, and at least two magnetic sensors arranged in a built-in space. The magnetic control device has a second magnet. IMU acquires a measurement value of the attitude, and at least two magnetic sensors acquire at least a measurement value of a first magnetic field and the measurement value of the second magnetic field. The processing device calculates the spin angle of the capsule endoscope, including a precisely estimated Pitch angle, a precisely estimated Roll angle, and an estimated Yaw angle. The processing device calculates the estimated position of the capsule endoscope relative to the second magnet.

### CROSS REFERENCE TO RELATED APPLICATIONS

This application is the United State national stage entry under 37 U.S.C. 371 of PCT/CN2021/091222, filed on Apr. 29, 2021, which claims priority to Chinese application number 202110290148.3, filed on Mar. 18, 2021, the disclosure of which are incorporated by reference herein in their entireties.

### FIELD OF THE DISCLOSURE

The disclosure relates generally to the field of medical devices. More specifically, the disclosure relates to capsule endoscope systems and magnetic positioning methods for capsule endoscopes of the systems.

### BACKGROUND

With the development of modern medical technology, pathological changes in the digestive cavity (such as polyps on the gastric wall) can be examined by introducing capsule endoscopy, which can help doctors obtain image information of polyps on the gastric wall to assist doctors in diagnosis and treatment of patients. Such capsule endoscopes can usually be subject to magnetic force, and doctors, nurses or other operators can conduct magnetic guidance on capsule endoscopes located in the digestive cavity by controlling external magnetic control devices, so that capsule endoscopes can move to the predetermined position in the digestive cavity for inspection. When the capsule endoscope is used to check the digestive cavity, in order to better control the capsule endoscope located in the digestive cavity, doctors need to know the specific location of the capsule endoscope in the digestive cavity.

In the prior art, the positioning methods of the capsule endoscope in the human body include ultrasonic positioning, radio frequency signal positioning, magnetic positioning, etc. The built-in magnetic positioning uses magnetic sensors and inertial measurement units, and uses error estimation algorithms to determine the position and the attitude of the capsule endoscope. However, the accuracy is not high.

### SUMMARY

The following presents a simplified summary of the invention in order to provide a basic understanding of some aspects of the invention. This summary is not an extensive overview of the invention. It is not intended to identify critical elements or to delineate the scope of the invention. Its sole purpose is to present some concepts of the invention in a simplified form as a prelude to the more detailed description that is presented elsewhere.

In some embodiments, the disclosure provides a capsule endoscope system including a capsule endoscope, a magnetic control device and a processing device. The capsule endoscope has a built-in space and a first magnet, an inertial measurement unit (IMU) and at least two magnetic sensors arranged in the built-in space, two of the at least two magnetic sensors are respectively close to two ends of the built-in space along the longitudinal direction. The magnetic control device has a second magnet, and the magnetic control device conducts magnetic control on the capsule endoscope through the action of the second magnet on the first magnet. The IMU senses the motion attitude of the capsule endoscope and acquires a measurement value of the attitude. The at least two magnetic sensors sense the magnetic field of the second magnet and acquire at least a measurement value of a first magnetic field and a measurement value of a second magnetic field. The processing device calculates the spin angle of the capsule endoscope based on the preset attitude solving algorithm and the measurement value of the attitude, and the spin angle includes a precisely estimated pitch angle, a precisely estimated roll angle and an estimated yaw angle. The processing device calculates the estimated position of the capsule endoscope relative to the second magnet based on the driving magnetic field algorithm, the measurement value of the first magnetic field, the measurement value of the second magnetic field and the spin angle, optimizes the estimated position and the estimated Yaw angle using the unscented Kalman Filter algorithm, acquires the precisely estimated position and Yaw angle of the capsule endoscope relative to the second magnet, and determines the position and the attitude of the capsule endoscope in the world coordinate system based on the position and the attitude of the second magnet.

In the capsule endoscope system of the disclosure, the number of sensors, especially the number of magnetic sensors, may be reduced by the combination of IMU and two magnetic sensors. The two magnetic sensors are arranged close to two longitudinal ends of the internal space of the capsule endoscope, which may make the distance between the two magnetic sensors as long as possible, thus increasing the accuracy of magnetic positioning. UKF algorithm is used for optimization. Its calculation method based on Bayesian theorem is convenient for fusing data from multiple sensors. It may not only deal with linear scenes, but also be applied to nonlinear scenes. It has fast calculation speed and may consider process error and measurement error at the same time, thus may be used to improve the positioning efficiency.

Optionally, the magnetic sensor is a triaxial measuring, digital output MEMS device. In this case, three axial magnetic field measurement values may be collected. The sensor may be made small in size and suitable for installation in capsule endoscopy using MEMS (Micro electro Mechanical Systems) devices. The number of special signal acquisition circuits may be reduced by adopting Digital output, and spaces further saved.

Optionally, the magnetic sensor placed closer to the first magnet of the at least two magnetic sensors is a Hall sensor. The Hall sensor has a large range, which may effectively solve the problem that the magnetic sensor is saturated due to its proximity to the first magnet.

Optionally, the magnetic sensor placed farther away from the first magnet of the at least two magnetic sensors is an anisotropic magnetoresistance (AMR) sensor. In this case, it may make full use of the characteristics of high sensitivity, small size and low power consumption of the AMR sensor.

Optionally, the IMU is optionally a six axis IMU. In this case, the six axis IMU may be used to more accurately measure the attitude of the capsule endoscope.

Optionally, the IMU optionally includes an accelerometer and a gyroscope. In this case, the linear acceleration of the capsule endoscope may be obtained through the accelerometer, and the angular velocity of the capsule endoscope may be obtained through the gyroscope, so that the attitude of the capsule endoscope may be obtained more accurately.

Optionally, the magnetic control device also has a coil which is matched with the second magnet to conduct magnetic control on the capsule endoscope, and when the magnetic control device conducts magnetic positioning on the capsule endoscope, the coil is in a non-working state. Thus, the interference to the magnetic field measurement of the capsule endoscope may be reduced, and the processing for removing the interference may be avoided, to further improve the positioning efficiency.

Optionally, the IMU and the at least two magnetic sensors have substantially the same coordinate orientation. In this case, the rotation transformation with large amount of calculation may be avoided, the calculation speed is accelerated, and the positioning efficiency may be improved.

Optionally, in the driving magnetic field algorithm the second magnet is equivalent to a magnetic dipole moment. In this case, when the second magnet is far from the capsule endoscope (more than 10 cm), the calculation may be simplified by the magnetic dipole moment model, so the results may be obtained quickly, which is conducive to real-time acquisition of positioning information.

In other embodiments, the disclosure provides a capsule endoscope magnetic positioning method of a capsule endoscope system including obtaining a measurement value of the attitude of the capsule endoscope. At a first position and a second position of the capsule endoscope, the magnetic field of the second magnet is sensed and a first magnetic field measurement value and a second magnetic field measurement value are obtained. The first position and the second position are respectively close to two ends along the longitudinal direction of the built-in space of the capsule endoscope, and the second magnet is used for magnetic control of the capsule endoscope. The spin angle of the capsule endoscope is calculated based on the preset attitude solution algorithm and the measurement value of the attitude. The spin angle includes a precisely estimated pitch angle, a precisely estimated roll angle and an estimated yaw angle. Based on the driving magnetic field algorithm and the measurement value of the first magnetic field, the measurement value of the second magnetic field and the spin angle, an estimated position of the capsule endoscope relative to the second magnet is calculated, and the estimated position and the estimated yaw angle are optimized using the unscented Kalman filter algorithm, a precisely estimated position and a precisely estimated yaw angle of the capsule endoscope relative to the second magnet are obtained. Based on the position and the attitude of the second magnet, the position and the attitude of the capsule endoscope in the world coordinate system are determined.

Optionally, in the magnetic positioning method according to the disclosure, the number of sensors, especially the number of magnetic sensors, may be reduced by adopting the measurement value of the attitude. The two measurement positions may be made as far away from each other as possible, thus increasing the accuracy of magnetic positioning, by obtaining the magnetic field measurement value of the second magnet close to the two longitudinal ends of the built-in space of the capsule endoscope respectively. UKF algorithm is used for optimization. Its calculation method based on Bayesian theorem is convenient for fusing data from multiple sensors. It may not only deal with linear scenes, but also be applied to nonlinear scenes. It has fast calculation speed and may consider process error and measurement error at the same time, thus may be used to improve the positioning efficiency.

Optionally, according to the capsule endoscope system and the magnetic positioning method of the capsule endoscope provided by the disclosure, when the external magnetic field is relatively strong, the capsule endoscope may be effectively positioned by processing the measured values of the internal IMU and the magnetic sensor of the capsule endoscope.

### BRIEF DESCRIPTION OF THE DRAWINGS

Illustrative embodiments of the disclosure are described in detail below with reference to the attached drawing figures.

FIG. 1 is a schematic diagram showing a capsule endoscope system according to an embodiment of the disclosure.

FIG. 2 is a schematic diagram showing the capsule endoscope according to an embodiment of the disclosure.

FIG. 3 is a schematic diagram showing the coordinate system conversion according to an embodiment of the disclosure.

FIG. 4 is a flowchart showing the magnetic positioning method of capsule endoscope of the capsule endoscope system according to an embodiment of the disclosure.

### DETAILED DESCRIPTION

The following describes some non-limiting exemplary embodiments of the invention with reference to the accompanying drawings. The described embodiments are merely a part rather than all of the embodiments of the invention. All other embodiments obtained by a person of ordinary skill in the art based on the embodiments of the disclosure shall fall within the scope of the disclosure.

It should be noted that the terms “include” and “have” in this disclosure and any variations thereof, such as the processes, methods, systems, products or devices that include or have a series of steps or units need not be limited to those steps or units that are clearly listed, but may include or have other steps or units that are not clearly listed or are inherent to these processes, methods, products or devices.

In addition, the subtitles involved in the following description of the disclosure are not intended to limit the content or scope of the disclosure, but only serve as a reminder for reading. Such subtitles may neither be understood as dividing the content of the article, nor should the content under the subtitle be limited to the scope of the subtitle.

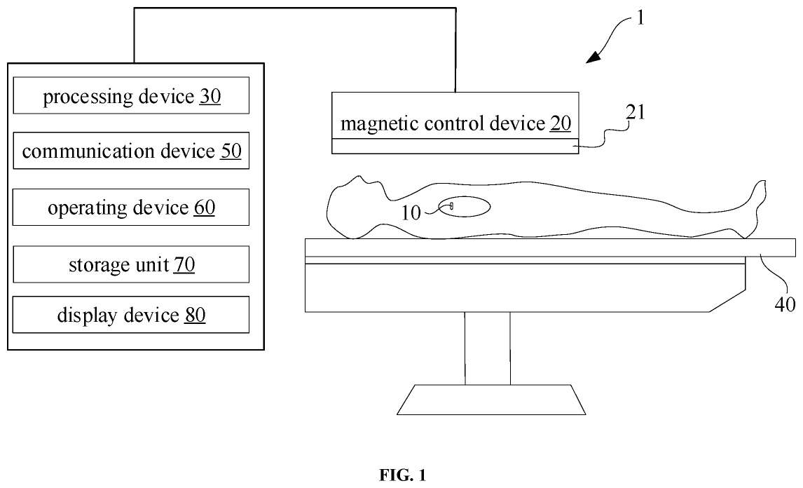

In FIGS. 1-4: 1 represents capsule endoscope system, 10 represents capsule endoscope, 11 represents first magnet, 12 represents IMU, 13 represents magnetic sensor, 131 represents first magnetic sensor, 132 represents second magnetic sensor, 14 represents first circuit board, 15 represents second circuit board, 16 represents third circuit board, 17 represents imaging device, 20 represents magnetic control device, 21 represents second magnet, 30 represents processing device, 40 represents examination table, 50 represents communication device, 60 represents operating device, 70 represents storage unit, and 80 represents display device.

Capsule endoscopy system is a medical instrument that uses capsule endoscopy to check the human digestive cavity. The system may help doctors diagnose digestive tract diseases of patients by monitoring the health status of gastrointestinal and esophageal parts of the human body through capsule endoscopy introduced into human body by for example swallowing.

A capsule endoscope system generally may include a capsule endoscope, a magnetic control device and a processing device. The capsule endoscope is a capsule shaped endoscope, which is easy to introduce into the human body to check the internal tissues with its camera device, and then discharged from the human body. The magnetic control device may use its magnet to conduct magnetic field action on the capsule endoscope, to drive the capsule endoscope to move in the human body according to the inspection requirements and obtain images of Volume of Interest (VOI). In order to realize the above drive, the magnetic control device needs to know the positioning information of the capsule endoscope in real time. The processing device may process the information returned by the sensor in the capsule endoscope, including the sensing information related to the position and the attitude, to obtain the position and the attitude of the capsule endoscope.

In some examples, the subject of the capsule endoscope system may be an animal, such as a human body. The part where capsule endoscope may be introduced into the tested body may be a tissue cavity such as a digestive cavity, such as stomach, esophagus, large intestine, colon, small intestine, etc. In addition, in some examples, it may also be a tissue cavity of a non-digestive cavity, such as an abdominal cavity, a chest cavity, and the like. For a digestive cavity, such as stomach, esophagus, large intestine, etc., the capsule endoscopy may enter the digestive cavity by eating, while for a non-digestive cavity, it may be placed into the non-digestive cavity by opening a minimally invasive opening through clinical surgery. Capsule endoscope system 1 is described in detail below, taking the stomach cavity as an example.

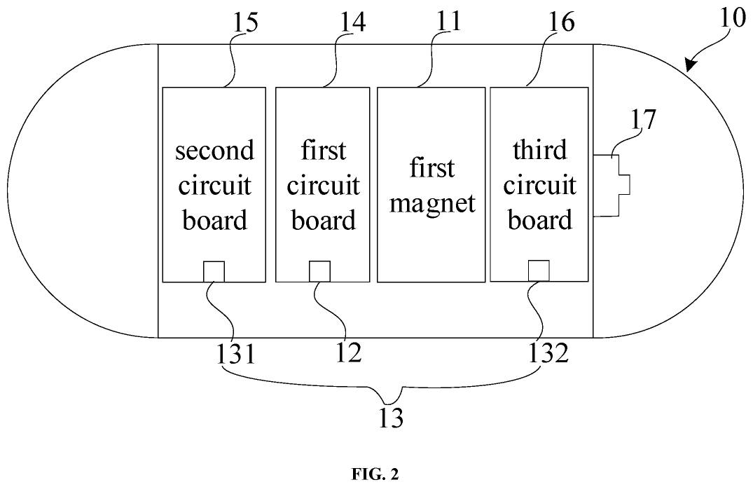

Capsule endoscope system 1 in the example of the disclosure may include capsule endoscope 10, magnetic control device 20 and processing device 30 (see FIG. 1). In this embodiment, capsule endoscope 10 may have first magnet 11 and may be introduced into the digestive cavity of the tested body (see FIG. 2), and magnetic control device 20 may have second magnet 21 and guide capsule endoscope 10 to move in the digestive cavity of the tested body through the action of second magnet 21 on first magnet 11.

In some examples, capsule endoscope 10 may be a medical device formed to be able to introduce into the tested body and shaped like a capsule. From the appearance, capsule endoscope 10 may be a capsule shaped shell (see FIG. 2) of a size that may be introduced into the inside of the tested body. The shell may be composed of a cylindrical shell and two dome shaped shells at the longitudinal ends of the cylindrical shell. The longitudinal openings at both ends of the cylindrical shell are plugged by the dome shaped shell to maintain a liquid tight state. The dome shaped shell is a transparent optical dome that transmits light (such as visible light) of a specified wavelength band. The cylindrical shell is generally opaque. The capsule type shell may enclose a built-in space for arranging relevant detection devices.

In some examples, as shown in FIG. 2, capsule endoscope 10 may include first magnet 11. First magnet 11 may be a permanent magnet, which may be arranged in the substantially middle position along the longitudinal direction of the internal space of capsule endoscope 10. Therefore, magnetic control device 20 may better control the position and the attitude of the capsule endoscope through the action of its second magnet 21 on first magnet 11.

In some examples, as shown in FIG. 2, capsule endoscope 10 may also include IMU 12. IMU 12 may be a combination of one or more sensors to obtain the attitude measurement information of capsule endoscope 10. In some examples, IMU 12 may be a six axis MEMS device in which an accelerometer and a gyroscope are packaged. In some examples, IMU12 may be soldered on first circuit board 14 (see FIG. 2). IMU12 may output the acceleration (aₓ, a_{y}, a_{z}) and the angular velocity (Wₓ, W_{y}, W_{z}) of capsule endoscope 10 at a certain time under the carrier coordinate system (i.e. the rectangular coordinate system where IMU 12 is located). The acceleration and the angular velocity respectively reflect the attitude of capsule endoscope 10 when it is stationary or moving. In this case, IMU 12 may obtain the measurement value of the attitude of capsule endoscope 10, so that part of the attitude information of capsule endoscope 10 may be solved, reducing the unknown amount to be solved, thus reducing the number of magnetic sensors 13.

In some examples, as shown in FIG. 2, capsule endoscope 10 may also include at least two magnetic sensors 13. Magnetic sensor 13 may obtain magnetic field measurement values of second magnet 21 and/or first magnet 11. In this embodiment, in order to simplify the sensor arrangement in the built-in space of capsule endoscope 10, magnetic sensor 13 may be set as two, namely first magnetic sensor 131 and second magnetic sensor 132. First magnetic sensor 131 may be close to the left end of the built-in space along the longitudinal direction of capsule endoscope 10, while second magnetic sensor 132 may be close to the right end of the built-in space along the longitudinal direction of capsule endoscope 10 (see FIG. 2). If the distance between magnetic sensors 13 is short from each other, the measured values of their respective output magnetic fields will also be close, which is approximately equivalent to a magnetic sensor as a whole. When the distance between first magnetic sensor 131 and second magnetic sensor 132 is long, the difference between the magnetic field measurement value of first magnetic sensor 131 and the magnetic field measurement value of second magnetic sensor 132 is large, which is conducive to effectively solving the amount to be solved. In this case, when the distance between magnetic sensors 13 is long enough, the positioning accuracy may meet the requirements. For example, when the distance interval is 2 cm, the positioning accuracy may reach about 5 mm. In this embodiment, first magnetic sensor 131 and second magnetic sensor 132 are close to the two ends of the built-in space respectively, so that they may be as far away from each other as possible within the allowable range of the built-in space, thus improving the positioning accuracy.

In some examples, there may be more than two magnetic sensors 13, such as three, which need to be arranged so that they are as far away from each other as possible. For example, two of three magnetic sensors 13 may be arranged close to the two ends along the longitudinal direction the built-in space of capsule endoscope 10, and the other may be arranged at a position substantially in the middle of the built-in space.

In some examples, sensors of the MEMS device may be selected as magnetic sensors 13. In this case, first magnetic sensor 131 and second magnetic sensor 132 may be respectively welded on second circuit board 15 and third circuit board 16 as chips (see FIG. 2). In some examples, the package size of first magnetic sensor 131 and second magnetic sensor 132 may be within 3*3 mm, and their outputs may be digital or analog outputs. In some examples, magnetic sensors 13 may output a digital signal in order to facilitate reading the magnetic field measurement value. In this case, it is not necessary to arrange a special signal acquisition circuit to collect the signal of magnetic sensors 13 in capsule endoscope 10, thus saving the space of the printed circuit board (PCB: Printed Circuit Board), thereby simplifying the design and process. Those skilled in the art should understand that the positions of second circuit board 15 and third circuit board 16, the position of first magnetic sensor 131 on second circuit board 15, and the position of second magnetic sensor 132 on third circuit board 16 in FIG. 2 are exemplary and are not limited thereto. Under the condition that the distance between first magnetic sensor 131 and second magnetic sensor 132 is long enough, second circuit board 15 arranged with first magnetic sensor 131 and third circuit board 16 arrangedwith second magnetic sensor 132 may also be arranged as following: second circuit board 15 is arranged far from first magnet 11, and third circuit board 16 is arranged near first magnet 11. Alternatively, second circuit board 15 is arranged close to first magnet 11, and third circuit board 16 is arranged far away from first magnet 11. Alternatively, second circuit board 15 is arranged away from first magnet 11, and third circuit board 16 is arranged away from first magnet 11. The position of first magnetic sensor 131 on second circuit board 15 and the position of second magnetic sensor 132 on third circuit board 16 may be arranged as following: first magnetic sensor 131 is at the lower part of second circuit board 15, and second magnetic sensor 132 is at the upper part of third circuit board 16. Alternatively, first magnetic sensor 131 is at the upper part of second circuit board 15 and second magnetic sensor 132 is at the lower part of third circuit board 16.

In some examples, a Hall sensor, an AMR sensor, or a tunnel magnetoresistance (TMR) sensor may be selected as magnetic sensors 13 to obtain magnetic field measurements of second magnet 21 and/or first magnet 11 (see FIG. 1). In some examples, because first magnetic sensor 131 is close to the left end of the built-in space along the longitudinal direction and is relatively far from first magnet 11 in the spatial layout, the AMR sensor may be used, which is based on the magnetoresistance effect that the resistance of the magnetic material changes with the external magnetic field, and has the characteristics of precision, high sensitivity, small size, low power consumption, etc., Therefore, the magnetic field measurement in case that first magnetic sensor 131 is far away from first magnet 11 may be conducted. Second magnetic sensor 132 is spatially arranged close to the end of the built-in space on the right side along the longitudinal direction, which is relatively close to first magnet 11. The Hall sensor may be used, which is based on the Hall principle, that is, the carrier in the semiconductor deflects under the magnetic field and generates a potential difference in the direction perpendicular to the current. The Hall sensor may be applied to a large number of magnetic field measurement occasions, and has the characteristics of large range, high sensitivity, small size, low power consumption, etc., which may meet the requirements of magnetic field measurement where second magnetic sensor 132 is closer to first magnet 11. Those skilled in the art should understand that the selection of magnetic sensors 13 is not limited to this, and the magnetic sensor that may effectively obtain the magnetic field measurement value of second magnet 21, reduce the risk of range saturation due to being close to first magnet 11, and meet the requirements for the internal space of the capsule endoscope may be used.

In some examples, IMU 12 and magnetic sensor 13 have substantially the same coordinate orientation, that is, the coordinate orientation (XYZ axis) of these devices is kept consistent for layout. In this case, the coordinate rotation transformation required for different device output data due to different coordinate orientations may be avoided, which reduces the complexity of the algorithm and improves the calculation efficiency.

In some examples, as shown in FIG. 2, capsule endoscope 10 may also include image pickup device 17 for image acquisition in the digestive cavity. Imaging device 17 may be arranged at the longitudinal end of the internal space of capsule endoscope 10. It captures VOI images through a transparent dome shaped shell at the end, and wirelessly transmits VOI images to related devices through circuit components and transmission antennas (not shown), such as communication device 50 (see FIG. 1).

In some examples, second magnet 21 of magnetic control device 20 may be a permanent magnet, and its magnetic moment is usually more than 1000 times larger than that of first magnet 11, which is also far higher than the environmental magnetic field, thus reducing the difficulty of magnetic field measurement of second magnet 21. When capsule endoscope 10 is introduced into the tested body, the distance between second magnet 21 and capsule endoscope 10 is usually more than 10 cm. Therefore, second magnet 21 may be equivalent to the magnetic dipole moment to simplify the calculation.

In some examples, magnetic control device 20 also has a coil (not shown). The coil assists second magnet 21 in magnetic control of capsule endoscope 10. Specifically, it mainly controls the jumping action of capsule endoscope 10. In order to reduce the interference of capsule endoscope 10 on the magnetic field measurement of second magnet 21, the coil may be in a non-operating state when the magnetic positioning is in an operating state. In some examples, the coil and the magnetic positioning may be in an alternate working state. In some necessary occasions, when magnetic positioning and coil are required to work at the same time, coil interference needs to be removed from the magnetic field measurement value of magnetic sensor 13.

In some examples, as shown in FIG. 1, processing device 30 may obtain the measurement value of the attitude of IMU 12, and use a preset attitude solving algorithm, such as a complementary filtering algorithm, to calculate the spin angle of capsule endoscope 10 relative to the world coordinate system. The spin angle may include precisely estimated Pitch angle, precisely estimated Roll angle and estimated Yaw angle. The estimated Yaw angle needs to be calibrated by the optimization algorithm described later.

In some examples, as shown in FIG. 1, processing device 30 may also obtain the magnetic field measurement value of magnetic sensors 13. Hereinafter, the driving magnetic field algorithm of second magnet 21 is described in detail in combination with the drawings.

FIG. 1 shows a scene where capsule endoscope system 1 is used for medical examination. At this time, the magnetic field measurement value obtained by magnetic sensors 13 is the superposition value of first magnet 11 and second magnet 21. In the non-inspection state, capsule endoscope 10 may be far away from second magnet 21 to obtain the magnetic field measurement value solely contributed by first magnet 11 through magnetic sensors 13. The magnetic field measurement value contributed by first magnet 11 is deducted from the superposition value, which is the magnetic field measurement value contributed by second magnet 21. The following magnetic field measurement values refer to the magnetic field measurement value contributed by second magnet 21. As mentioned above, the distance between second magnet 21 and capsule endoscope 10 is usually more than 10 cm. In this case, second magnet 21 may be equivalent to the magnetic dipole moment, and the relevant amount may be calculated using the following equation (1).

$$\vec{B} = \frac{{μ}_{0}}{4 π} ( \frac{3 ( \vec{m} \cdot \vec{r} ) \vec{r}}{{r}^{5}} - \vec{\frac{m}{{r}^{3}}} ) \qquad ( 1 )$$

In formula (1), \vec{m} is the magnetic moment of second magnet 21 and its value (mode of \vec{m}) is a known quantity, \vec{B} is the magnetic field measurement value of magnetic sensor 13, μ₀ is the vacuum permeability, and \vec{r} is the quantity to be measured.

Taking first magnetic sensor 131 and second magnetic sensor 132 as examples, the process that processing device 30 calculates the position and the attitude of capsule endoscope 10 will be explained hereinafter.

In some examples, first magnetic sensor 131 obtains a first magnetic field measurement value, and second magnetic sensor 132 obtains a second magnetic field measurement value. Processing device 30 calculates the estimated position of capsule endoscope 10 relative to second magnet 21 by formula (1) based on the measurement value of the first magnetic field and the measurement value of the second magnetic field. In some examples, the magnetic field measurement error of second magnet 21(that is, the magnetic field measurement error of the measurement value of the first magnetic field and the magnetic field measurement error of the measurement value of the second magnetic field) may be calculated, and the magnetic field measurement error may be optimized. In some examples, nonlinear least square method and particle swarm optimization (PSO) algorithm may be used to optimize the magnetic field measurement error. In some examples, UKF algorithm may be used to optimize the above estimated position and estimated Yaw angle based on the measurement value of the first magnetic field, the measurement value of the second magnetic field and the spin angle obtained by IMU 12. The UKF algorithm assumes that the observation data is Gaussian distributed. Based on Bayesian theorem, it calculates the maximum likelihood probability distribution under a posteriori condition. Compared with the nonlinear least squares method and PSO algorithm, it is more efficient and easier to integrate other data. It is not only applicable to linear scenes, that is, the state equation and the observation equation are linear, but also adaptive to nonlinear scenes. It also takes process error and measurement error into account. In some examples, when using the UKF algorithm for optimization, the magnetic field measurements of each sensor in magnetic sensor 13 may be time stamped uniformly. Processing device 30 uses the UKF to optimize the results to output the precisely estimated position and precisely estimated Yaw angle of capsule endoscope 10 relative to second magnet 21. Together with the precisely estimated Pitch angle and precisely estimated Roll angle obtained previously, the precisely estimated position and precisely estimated attitude of capsule endoscope 10 relative to second magnet 21 may be obtained.

In some examples, after obtaining the precisely estimated attitude of capsule endoscope 10 in the world coordinate system and the precisely estimated position relative to second magnet 21 through the above calculation, processing device 30 fuses the position and the attitude of second magnet 21 in the world coordinate system, calculates the rotation matrix from capsule endoscope 10 coordinate system to the world coordinate system, then obtains the position and the attitude of capsule endoscope 10 in the world coordinate system.

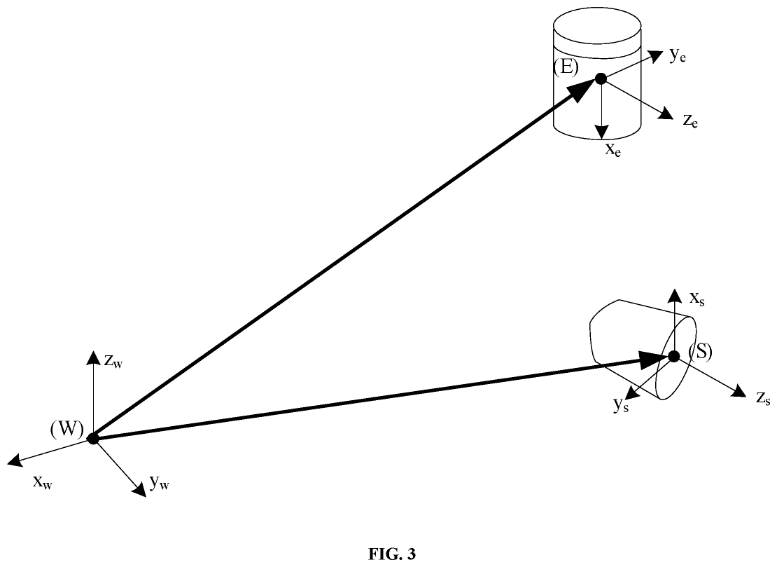

FIG. 3 is a schematic diagram showing the coordinate system conversion involved in the example of the disclosure. As shown in FIG. 3, (W) represents the world coordinate system, where x_{w}, y_{w} and z_{w} are the three coordinate axes of the world coordinate system respectively. (S) Represents the coordinate system of capsule endoscope 10, where xₛ, yₛ and zₛ are the three coordinate axes of the coordinate system of capsule endoscope 10. (E) Represents the coordinate system of second magnet 21. Here, xₑ, yₑ and zₑ are the three coordinate axes of second magnet 21 coordinate system respectively.

In some examples, as shown in FIG. 1, capsule endoscope system 1 may also include examination table 40 for carrying the subject, communication device 50 for wireless communication with capsule endoscope 10 in the subject, operating device 60 for controlling magnetic control device 20 and examination table 40, storage unit 70 for storing various information such as VOI images of the subject and display device 80 that displays various information such as the VOI image of the tested body collected by capsule endoscope 10.

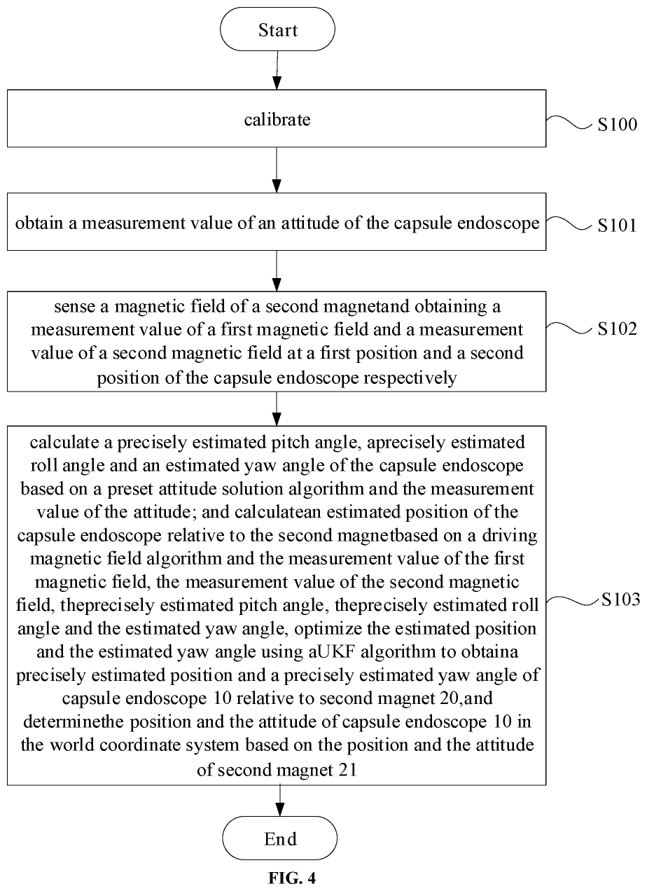

FIG. 4 is a flowchart showing the magnetic positioning of the capsule endoscope using the capsule endoscope system of the example of the disclosure. In some examples, as shown in FIG. 3, in combination with FIGS. 1 and 2, the principle of magnetic positioning using the capsule endoscope system involved in the examples of the disclosure may include acquiring the measurement value of the attitude of capsule endoscope 10 (step S101). In some examples, the measurement value of the attitude may be obtained by IMU 12 arranged in the built-in space of capsule endoscope 10 (see FIG. 2). The magnetic field of second magnet 21 is induced at the first position and the second position of capsule endoscope 10, respectively, and the measurement value of the first magnetic field and the measurement value of the second magnetic field are obtained (step S102). In some examples, the measurement value of the first magnetic field and the measurement value of the second magnetic field may be obtained through first magnetic sensor 131 and second magnetic sensor 132 in capsule endoscope 10. The first position and the second position are respectively close to two ends of the inner space of capsule endoscope 10 along the longitudinal direction. In some examples, capsule endoscope 10 may be magnetically controlled by second magnet 21 provided by magnetic control device 20. Based on the preset attitude solution algorithm and the measurement value of the attitude, the precisely estimated Pitch angle, the precisely estimated Roll angle and the estimated Yaw angle of capsule endoscope 10 are calculated. Based on the driving magnetic field algorithm, the measurement value of the first magnetic field, the measurement value of the second magnetic field, the precisely estimated pitch angle, the precisely estimated roll angle and the estimated Yaw angle, the estimated position of capsule endoscope 10 relative to second magnet 21 is calculated, and the UKF algorithm is used to optimize the estimated position and the estimated Yaw angle to obtain the precise estimated position and the precise Yaw angle of capsule endoscope 10 relative to second magnet 21. Based on the position and the attitude of second magnet 21, the position and the attitude of capsule endoscope 10 in the world coordinate system are determined (step S103). In some examples, the position and the attitude of capsule endoscope 10 in the world coordinate system may be calculated by processing device 30.

In some examples, calibration may also be included before step S101 (step S100).

In step S100, capsule endoscope 10 may be placed in an environment far away from magnetic control device 20, and the background magnetic field may be obtained by horizontally rotating one circle according to the common inertial navigation calibration method. The background magnetic field may include the geomagnetic field and the magnetic field of first magnet 11. The background magnetic field will be deducted from the subsequent measured values to eliminate its interference.

In step S101, the acceleration and the angular velocity of capsule endoscope 10 may be measured. Capsule endoscope 10 may be introduced into the body to work normally. As described above, IMU 12 arranged in the internal space of capsule endoscope 10 may obtain the measurement value of the attitudes of capsule endoscope 10, that is, the acceleration and the angular velocity of capsule endoscope 10, and transmit them to processing device 30, for example, to the PC terminal where processing device 30 is located in text mode.

In step S102, referring to FIG. 2, first magnetic sensor 131 is arranged near the first position at the left end of capsule endoscope 10, and second magnetic sensor 132 is arranged near the second position at the right end of capsule endoscope 10. Second magnet 21 contributes to the magnetic field measurement value obtained by the two magnetic sensors after deducting the background magnetic field.

In some examples, IMU 12 and magnetic sensor 13 may be required to have a high sampling rate, such as 100 Hz. All sensors have a uniform time stamp. In some examples, the delay caused by wireless transmission may also be required to be as low as possible to meet the real-time requirements.

In step S103, based on the measurement value of the attitude of IMU 12, processing device 30 calculates the spin angle of capsule endoscope 10 using a preset attitude solution algorithm, such as a complementary filtering algorithm. In some examples, the spin angle may include precisely estimated Pitch angle, the precisely estimated Roll angle and the estimated Yaw angle. Processing device 30 uses the driving magnetic field algorithm to calculate the estimated position of capsule endoscope 10 relative to second magnet 21 based on the measurement value of the first magnetic field of first magnetic sensor 131 and the measurement value of the second magnetic field of second magnetic sensor 132. In some examples, because the distance between second magnet 21 and capsule endoscope 10 is long (more than 10 cm), the driving magnetic field algorithm in which second magnet 21 is equated to a magnetic dipole moment may be used. Specifically, equation (1) may be used for related calculation. Then, UKF algorithm is used to optimize the estimated position and estimated Yaw angle based on the measured value of the first magnetic field, the measured value of the second magnetic field and the spin angle to obtain the precisely estimated position and Yaw angle of capsule endoscope 10 relative to second magnet 21. Processing device 30 may fuse the position and the attitude of second magnet 21 in the world coordinate system, calculate the rotation matrix from capsule endoscope 10 coordinate system to the world coordinate system, and obtain the position and the attitude of capsule endoscope 10 in the world coordinate system, as shown in FIG. 3.

According to the disclosure, the magnetic positioning efficiency of the capsule endoscope in the capsule endoscope system may be improved.

Although the disclosure has been specifically described above in combination with the drawings and examples, it is understood that the above description does not limit the disclosure in any form. Those skilled in the art may, as required, make changes to the disclosure without departing from the essential spirit and scope of the disclosure, and these changes fall within the scope of the disclosure.

Various embodiments of the disclosure may have one or more of the following effects. In some embodiments, the disclosure may be proposed in view of the state of the prior art, and may provide a capsule endoscope system and its magnetic positioning method capable of improving the magnetic positioning accuracy. In other embodiments, the disclosure may also describe a magnetic positioning method of the capsule endoscope, where the magnetic positioning efficiency may be improved by the UKF algorithm for optimization. In further embodiments, the processing device may calculate the estimated position of the capsule endoscope relative to the second magnet, and may use the UKF algorithm to obtain the precisely estimated position and the precisely estimated Yaw angle of the capsule endoscope relative to the second magnet, to obtain the position and the attitude of the capsule endoscope in the world coordinate system.

While the disclosure has been particularly shown and described with reference to the accompanying drawings and examples, it is to be understood that the disclosure is not limited in any manner by the foregoing description. It will be apparent to those skilled in the art that various modifications and variations may be made to the disclosure without departing from the spirit or scope of the disclosure.

Many different arrangements of the various components depicted, as well as components not shown, are possible without departing from the spirit and scope of the disclosure. Embodiments of the disclosure have been described with the intent to be illustrative rather than restrictive. Alternative embodiments will become apparent to those skilled in the art that do not depart from its scope. A skilled artisan may develop alternative means of implementing the aforementioned improvements without departing from the scope of the disclosure.

It will be understood that certain features and subcombinations are of utility and may be employed without reference to other features and subcombinations and are contemplated within the scope of the claims. Unless indicated otherwise, not all steps listed in the various figures need be carried out in the specific order described.

1-10. (canceled)

11. A capsule endoscope system comprising a capsule endoscope, a magnetic control device, and a processing device, wherein:
the capsule endoscope comprises a built-in space and a first magnet, an inertial measurement unit, and at least two magnetic sensors arranged in the built-in space, two of the at least two magnetic sensors being respectively close to two ends of the built-in space along a longitudinal direction;
the magnetic control device comprises a second magnet, and the magnetic control device conducts magnetic control on the capsule endoscope through an action of the second magnet on the first magnet;
the inertial measurement unit senses an attitude of the capsule endoscope and acquires a measurement value of the attitude, and the at least two magnetic sensors sense a magnetic field of the second magnet and acquire at least a measurement value of a first magnetic field and another measurement value of a second magnetic field; and
the processing device calculates a spin angle of the capsule endoscope based on a preset attitude solving algorithm and the measurement value of the attitude, wherein:
the spin angle comprises a precisely estimated pitch angle, a precisely estimated roll angle, and an estimated yaw angle, and
the processing device calculates an estimated position of the capsule endoscope relative to the second magnet based on a driving magnetic field algorithm, the measurement value of the first magnetic field, the measurement value of the second magnetic field and the spin angle, optimizes the estimated position and the estimated yaw angle using an unscented Kalman filtering algorithm to obtain a precisely estimated position and a precisely estimated yaw angle of the capsule endoscope relative to the second magnet, and determines a position and the attitude of the capsule endoscope in a world coordinate system based on the position and the attitude of the second magnet.

12. The capsule endoscope system according to claim 11, wherein the at least two magnetic sensors comprise a triaxial measuring, digital output MEMS device.

13. The capsule endoscope system according to claim 12, wherein a magnetic sensor placed closer to the first magnet of the at least two magnetic sensors is a Hall sensor.

14. The capsule endoscope system according to claim 12, wherein a magnetic sensor placed farther away from the first magnet of the at least two magnetic sensors is an anisotropic magnetoresistance (AMR) sensor.

15. The capsule endoscope system according to claim 11, wherein the inertial measurement unit is a six axis inertial measurement unit.

16. The capsule endoscope system according to claim 15, wherein the inertial measurement unit comprises an accelerometer and a gyroscope.

17. The capsule endoscope system according to claim 11, wherein:
the magnetic control device comprises a coil matched with the second magnet to conduct magnetic control on the capsule endoscope; and
when the magnetic control device conducts a magnetic positioning on the capsule endoscope, the coil is in a non-working state.

18. The capsule endoscope system according to claim 17, wherein the inertial measurement unit and the at least two magnetic sensors have a substantially same coordinate orientation.

19. The capsule endoscope system according to claim 11, wherein, in the driving magnetic field algorithm, the second magnet is equivalent to a magnetic dipole moment.

20. A capsule endoscope magnetic positioning method, comprising:
obtaining a measurement value of an attitude of a capsule endoscope;
sensing a magnetic field of a second magnet and obtaining a measurement value of a first magnetic field and another measurement value of a second magnetic field at a first position and a second position of the capsule endoscope respectively, wherein the first position and the second position are respectively close to two ends along a longitudinal direction of a built-in space of the capsule endoscope, and the second magnet is used in magnetic control for the capsule endoscope;
calculating a spin angle of the capsule endoscope based on a preset attitude solution algorithm and the measurement value of the attitude, wherein the spin angle comprises a precisely estimated pitch angle, a precisely estimated roll angle, and an estimated yaw angle;
calculating an estimated position of the capsule endoscope relative to the second magnet based on a driving magnetic field algorithm and the measurement value of the first magnetic field, the measurement value of the second magnetic field, and the spin angle;
optimizing the estimated position and the estimated yaw angle using an unscented Kalman filter algorithm to obtain a precisely estimated position and a precisely estimated yaw angle of the capsule endoscope relative to the second magnet; and
determining a position and the attitude of the capsule endoscope in a world coordinate system based on a position and the attitude of the second magnet.

21. The capsule endoscope magnetic positioning method according to claim 20, wherein:
the capsule endoscope comprises a built-in space and a first magnet; and
at least two magnetic sensors are provided at the first position and at the second position respectively.

22. The capsule endoscope magnetic positioning method according to claim 21, wherein the at least two magnetic sensors comprise a triaxial measuring, digital output MEMS device.

23. The capsule endoscope magnetic positioning method according to claim 21, wherein a magnetic sensor placed closer to the first magnet of the at least two magnetic sensors is a Hall sensor.

24. The capsule endoscope magnetic positioning method according to claim 21, wherein a magnetic sensor placed farther away from the first magnet of the at least two magnetic sensors is an anisotropic magnetoresistance (AMR) sensor.

25. The capsule endoscope magnetic positioning method according to claim 21, wherein an inertial measurement unit is used to obtain the measurement value of the attitude of the capsule endoscope.

26. The capsule endoscope magnetic positioning method according to claim 25, wherein the inertial measurement unit is a six axis inertial measurement unit.

27. The capsule endoscope magnetic positioning method according to claim 26, wherein the inertial measurement unit comprises an accelerometer and a gyroscope.

28. The capsule endoscope magnetic positioning method according to claim 25, wherein the inertial measurement unit and the at least two magnetic sensors have a substantially same coordinate orientation.

29. The capsule endoscope magnetic positioning method according to claim 20, further comprising using a coil matched with the second magnet to conduct magnetic control on the capsule endoscope, wherein, when magnetic positioning is conducted on the capsule endoscope, the coil is in a non-working state.

30. The capsule endoscope magnetic positioning method according to claim 20, wherein, in the driving magnetic field algorithm, the second magnet is equivalent to a magnetic dipole moment.
